(19) United States
(12) Patent Application Publication
(10) Pub. No.: US 2010/0189716 A1
(43) Pub. Date: Jul. 29, 2010

(54) TREATMENT OF HODGKINS LYMPHOMA

(75) Inventors: Arturo Molina, San Diego, CA (US); Kandasamy Hariharan, San Diego, CA (US); Steffan Ho, San Diego, CA (US)

Correspondence Address: PILLSBURY WINTHROP SHAW PITTMAN LLP, P.O. BOX 10500, MCLEAN, VA, 22102, US

(73) Assignee: BIOGEN IDEC INC., Cambridge, MA (US)

(21) Appl. No.: 12/593,281

(22) PCT Filed: Mar. 28, 2008
(86) PCT No.: PCT/US08/58660

§ 371 Date: Feb. 25, 2010

Related U.S. Application Data
(60) Provisional application No. 60/908,646, filed on Mar. 28, 2007.

(51) Int. Cl. A61K 39/395; C12N 5/00; A61P 35/00

(52) U.S. Cl. 424/133.1; 424/145.1; 424/158.1; 435/375

(57) ABSTRACT

Compositions comprising CD80 antagonists and methods using these compositions are provided for the treatment of Hodgkins lymphoma. More particularly, the disclosed CD80 antagonists may be used to induce apoptosis or lysis of Hodgkins Reed-Sternberg (HRS) cells, or to inhibit HRS cell activities that promote tumor development or progression.

### RELATED APPLICATIONS

Priority is claimed from U.S. Provisional Application No. 60/908,646, filed Mar. 28, 2007, which is incorporated herein by reference in its entirety.

### FIELD OF THE INVENTION

Compositions comprising CD80 antagonists and methods using these compositions are provided for treatment of Hodgkins lymphoma. More particularly, the disclosed CD80 antagonists may be used to induce apoptosis or lysis of Hodgkins Reed-Sternberg (HRS) cells, or to inhibit HRS cell activities that promote tumor development or progression.

### BACKGROUND OF THE INVENTION

Hodgkin lymphoma (HL) is an uncommon malignancy with 7,800 new cases and 1,490 deaths currently projected each year. Although many patients achieve durable remissions to front-line therapy, up to 40% of patients with advanced disease or poor prognostic features relapse. Although these patients may be treated with high dose chemotherapy and/or autologous stem cell rescue, many of these patients still succumb to the disease. Furthermore, many patients do not have the option of allogeneic transplant due to a lack of appropriate donors. Accordingly, there is a need in the art for additional treatment options for patients with relapsed or refractory Hodgkins lymphoma.

The present invention provides methods of treating Hodgkins lymphoma by administering to a subject in need thereof a therapeutically effective dose of a CD80 antagonist, such as, a small molecule CD80 antagonist or an anti-CD80 antibody. Also provided are various combination therapies for treating Hodgking's lymphoma, in which a CD80 antagonist and one or more additional therapeutic agents are administered simultaneously or sequentially in any order to thereby elicit a synergistic therapeutic effect. Representative additional therapeutic agents include an anti-CD30 antibody, an anti-CD40 antibody, an anti-RANK antibody, an anti-RANKL antibody, an anti-TRAIL antibody, an anti-Notch antibody, an anti-LMP antibody, an anti-IL-13 antibody, an anti-CD20 antibody, an anti-CD52 antibody, a CCR4 antibody, and antigen-binding fragments thereof. Additional agents useful in the disclosed combination therapies include proteosome inhibitors and histone deacytylase inhibitors.

In other aspects of the invention, methods are provided for inducing lysis of a Hodgkins Reed Sternberg cell by antibody-dependent-cellular-cytotoxicity by contacting the Hodgkins Reed Sternberg cell with an anti-CD80 antibody. Also provided are methods of inducing apoptosis or reducing cell growth of a Hodgkins Reed Sternberg cell by contacting a cell in a surrounding cellular infiltrate of a Hodgkins Reed Sternberg cell with a CD80 antagonist as described herein.

### BRIEF DESCRIPTION OF THE DRAWINGS

FIGS. 1A-1F show the results of flow cytometric analysis using propidium iodide (left trace) and a phycoerythrin (PE)-labeled anti-CD80 antibody (right trace) on four Hodgkins lymphoma cell lines (FIGS. 1A-1D) and two control B cell lymphoma cell lines (FIGS. 1E-1F). PEMB, number of PE molecules bound; MFI, mean fluorescent intensity.

FIGS. 2A-2F show the results of flow cytometric analysis determining CD80, CD86, CD30 and CD19 expression on L-428 cells (FIGS. 2A-2C) and Ramos lymphoma cells (FIGS. 2D-2F).

FIG. 3 is a line graph showing antibody dependent cellular cytotoxicity of L-428 Hodgkins lymphoma cells using the indicated antibodies.

FIG. 4 shows the results of inhibition of interleukin-6 production by L-428 cells cultured in the presence of galiximab and CTLA4-Ig.

### DETAILED DESCRIPTION

The invention provides compositions and methods comprising CD80 antagonists to induce apoptosis or to inhibit cell growth of Hodgkins Reed Sternberg (HRS) cells. The methods comprise targeting HRS cells, or cells that constitute an infiltrate surrounding the HRS cells, with a CD80 antagonist. The invention also provides methods and compositions for killing HRS cells or cells of the surrounding cellular infiltrate via antibody-dependent-cellular-cytotoxicity (ADCC). As disclosed herein, cell growth inhibition of HRS cells can also result from blockade of CD80 dependent cytokine/growth factor production in the surrounding cell infiltrates, for example, by T cells and macrophages, which support survival and progression of Hodgkins lymphoma cells. These compositions and methods may be used to treat Hodgkins lymphoma.

### I. Hodgkins Reed Sternberg Cells

HRS cells are found in Hodgkins lymphoma, other B cell lymphomas, such as lymphocytic leukemia (B-CLL) and in infectious diseases, such as infectious mononucleosis. HRS cells serve as a diagnostic marker for Hodgkins lymphoma, and may be necessary to the survival of Hodgkins lymphoma tumors. Relatively recently, HRS cells were recognized as being derived from B cells (Kuppers et al. Proc. Natl. Acad. Sci. USA., 1994, 91:10962-10966). This finding resulted in the WHO reclassification of Hodgkins disease to Hodgkins lymphoma (Harris, Mod. Pathol., 1999, 12:159-175). HRS cells, however, are phenotypically distinguishable from B cells, having diameters greater than 50 μm, are mono- or multi-nucleated, and have clearly visible nucleoli in each nucleus. Additionally, HRS cells do not express B cell markers such as CD20 or SYK, but almost universally express CD30 markers, which are rarely expressed in normal cells.

In Hodgkins lymphoma, the HRS cells are suspended in an abundant cellular infiltrate and represent only 1% or less of all cells in a Hodgkins lymphoma tumor. This cellular infiltrate is predominantly composed of CD4⁺ T lymphocytes. Other cell types include CD20⁺ B cells, CD8⁺ T cells, monocytes, eosinophils and dendritic cells. HRS cells secrete a cascade of cytokines and chemokines, which act to attract the surrounding immune cells of the infiltrate. For example, HRS cells express several chemokines such as thymus- and activation-regulated chemokine, eotaxin, macrophage-derived chemokine, inducible protein-10 and interleukin-8. The receptors for these chemokines are predominantly expressed on the cells of the surrounding infiltrate. Ligands secreted by the immune cells may act as survival or anti-apoptotic signals to HRS cells. For example, HRS cells express CD40 receptors, which when bound to CD40 ligand that is expressed by cells in the surrounding infiltrate, activate the apoptosis-inhibiting NFκB transcription factor. Thus, the surrounding infiltrate contains cells that enable the HRS cells to survive.

The compositions and methods of the present invention are useful for inducing apoptosis or reducing growth of HRS cells by blocking, inhibiting or diminishing the survival or anti-apoptotic signals, (i.e., receptors, cytokines, chemokines) that are expressed or secreted by the cells in the surrounding infiltrate. The compositions and methods of the invention are also useful for inducing apoptosis of HRS cells by blocking, inhibiting or diminishing the anti-apoptotic signals or survival signals that are expressed by the HRS cells themselves. For example, HRS cells express T helper (Th)1- and Th2-type cytokines, including interleukin (IL)-2, -4, -5, -6, -9, -10, -12, -13 and -15. HRS cells may express both the cytokine and its receptor, creating autocrine loops that act to support HRS cell survival. Accordingly, the compositions and methods of the invention also act to block, inhibit or diminish these HRS cell-derived signals. The blocking, inhibiting or diminishing of survival or anti-apoptotic signals may be accomplished at a molecular level or at a cellular level.

Among the cells of the surrounding infiltrate are regulatory T cells, which suppress the activation and/or proliferation of effector T cells. The regulatory T cells are CD4⁺, CD25⁺ and may be further characterized by expression of CC chemokine receptor 4 (CCR4) and/or forkhead box p3 (FOXP3). CD80 antagonists that deplete regulatory T cells, or which block, inhibit, or downregulate regulatory T cell functions are useful in the disclosed therapeutic methods. Regulatory T cells require CD28 co-stimulation, and therefore, useful CD80 antagonists includes molecules that block CD80/CD28 signaling, with or without blockade of B7/CTLA-4 signaling. Selective blockade of CD80/CD28 signaling may offer improved therapeutic efficacy given that the negative modulation of immune responses by B7/CTLA4 signaling is not disrupted.

II. CD80 Antagonists and their Use in Modulating HRS Cells

The present HRS cell modulating compositions of the invention comprise CD80 antagonists alone or in combination with other HRS modulating compositions as described below. CD80, a member of the B7 family of immune co-stimulatory molecules, is a 60-kDa transmembrane glycoprotein, which is expressed on HRS cells as well as cells in the surrounding HRS infiltrate (Delabie et al., Blood, 1993, 82: 2845-2852; Munro et al., Blood, 1994, 83: 793-798). These surrounding infiltrate cells include B cells and macrophages. CD80 is also expressed on professional antigen presenting cells and is the natural ligand for CD28 on T cells. Binding of CD80 to CD28 provides a co-stimulatory signal for T cell activation after engagement of the T cells receptor, leading to T cell proliferation and the secretion of cytokines including interleukin-2 (IL-2), tumor necrosis factor α (TNFα) and γ-interferon (IFNγ). Along with CD86, CD80 molecules provide the necessary stimuli to prime T cells against antigens presented by antigen presenting cells. Because HRS cells are postulated to act as antigen presenting cells (Murray et al. J. Clin. Pathol., 1995, 48: M105-M108), HRS cells may activate T cells, thereby inducing cytokine expression which aid in their survival.

The CD80 antagonists of the invention encompass molecules that may destroy a cell, such as an HRS cell or a cell in the surrounding infiltrate, upon binding to a CD80 antigen expressed by the cell. Alternatively or additionally, the CD80 antagonist may deplete the cell within a subject treated with the CD80 antagonist. Alternatively or additionally, the CD80 antagonist may interfere with one or more of the cell's functions, for example, the CD80 antagonist may by reduce the amount of cytokine secreted from the cell or prevent cytokines from being secreted. The CD80 antagonists may also act to deplete or destroy CD80-expressing cell via various mechanisms such as induction of apoptosis, inhibition or reduction of cell growth and/or antibody-dependent cell-mediated cytotoxicity (ADCC).

II.A. Anti-CD80 Antibodies

CD80 antagonists of the present invention include anti-CD80 antibodies or CD80-binding fragments thereof. As is well known in the art, naturally occurring antibodies comprise two identical light polypeptide chains of a molecular weight of approximately 23,000 Daltons, and two identical heavy chains of a molecular weight of 53,000-70,000 Daltons. The four chains are joined by disulfide bonds in a Y configuration, wherein the light chains bracket the heavy chains starting at the mouth of the Y. The variable domains of both the light (V_{L}) and heavy (V_{H}) chains determine antigen recognition and specificity. Conversely, the constant domains of the light chain (C_{L}) and the heavy chain (C_{H}1, C_{H}2 or C_{H}3) confer biological properties such as Fc receptor binding, complement binding and the like. Anti-CD80 antibodies useful in the present invention, may include polyclonal antibodies, monoclonal antibodies (mAbs), humanized or chimeric antibodies, PRIMATIZED® antibodies, which contain human constant regions and primate (cynomolgus macaque) variable regions, human monoclonal antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may have a structure of a naturally occurring antibody, multivalent forms thereof, or fragments thereof.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 1975, 256:495-497; and U.S. Pat. No. 4,376,110), the human B cell hybridoma technique (Kosbor et al., Immunology Today, 1983, 4:72; Cote et al., Proc. Natl. Acad. Sci. USA, 1983, 80:2026-2030), and the EBV-hybridoma technique (Cole et al., in Monoclonal Antibodies And Cancer Therapy, 1995, Alan R. Liss, Inc., New York, pp. 77-96). Such antibodies may be of any immunoglobulin class including IgG, IgM, IgE, IgA, IgD and any subclass thereof. Hybridomas producing monoclonal antibodies may be cultivated in vitro or in vivo.

A chimeric antibody is a molecule in which different portions are derived from different animal species, such as those having a variable region derived from a murine mAb and a human immunoglobulin constant region. Chimeric antibodies may be produced (Morrison et al., Proc. Natl. Acad. Sci. USA, 1984, 81:6851-6855; Takeda et al., Nature, 1985, 314:452-454) by splicing the genes from a mouse antibody molecule of appropriate antigen specificity together with genes from a human antibody molecule of appropriate biological activity.

A humanized antibody is a type of chimeric antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. These general approaches may be combined with standard mutagenesis and synthesis techniques to produce an HRS modulating antibody of any desired sequence.

The antibodies of the present invention may also be human monoclonal antibodies, for example those produced by immortalized human cells, by SCID-hu mice or other non-human animals capable of producing human antibodies. Human antibodies may also be isolated from antibody phage libraries, for example, as described by Marks et al., J. Mol. Biol., 1991, 222:581-597. Chain shuffling and recombination techniques may be used to produce phage libraries having increased antibody diversity, e.g., libraries including antibodies with increased binding affinity. See Marks et al., Biotechnology, 1992, 10:779-783 and Waterhouse et al., Nuc. Acids. Res., 1993, 21:2265-2266.

The antibodies useful in the present invention can also comprise single chain antibodies, which are typically formed by linking the heavy and light chain fragments of the Fv region via an amino acid bridge, resulting in a single chain polypeptide. See e.g., U.S. Pat. No. 4,946,778; Bird, Science, 1988, 242:423-426; Huston et al., Proc. Natl. Acad. Sci. USA, 1988, 85:5879-83; and Ward et al., Nature, 1989, 334:544-546).

Antibodies useful in the invention also include non-fucosylated antibodies. Such antibodies include an Fc region and complex N-glycoside-linked sugar chains bound to the Fc region, wherein among the total complex N-glycoside-linked sugar chains bound to the Fc region, the ratio of a sugar chain in which fucose is not bound to N-acetylglucosamine in the reducing end in the sugar chain is at least 20%. The non-fucosylated antibodies have enhanced Fc receptor binding and enhanced effector functions as compared to control fucosylated antibodies. See U.S. Provisional Application No. 60/908,643, filed Mar. 28, 2007, which is incorporated herein by reference in its entirety. Representative methods for production of non-fucosylated antibodies are set forth in Example 1. See also U.S. Patent Publication No. 2004/0093621, U.S. Pat. No. 6,946,292, PCT International Publication No. WO 2006/089232, and European Published Application No. 1176195, each of which is also incorporated herein by reference in its entirety.

Antibody fragments that recognize specific antigens or surface receptors as described herein may be generated by known techniques. For example, such fragments include F(ab′)₂ fragments or Fc regions that can be produced by pepsin digestion of the antibody molecule and Fab fragments that can be generated by reducing the disulfide bridges of the F(ab′)₂ fragments. Alternatively, Fab expression libraries may be constructed (Huse et al., Science, 1989, 246:1275-1281) to allow rapid and easy identification of monoclonal Fab fragments with the desired specificity.

Specific binding of an antibody or antibody fragment to the antigens described herein refers to preferential binding of an antibody to the antigen in a heterogeneous sample comprising multiple different antigens. Substantially lacking binding describes a level of binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

A representative anti-CD80 useful in the invention is galiximab, which is alternatively referred to as PRIMATIZED® 16C10, IDEC-114, or a PRIMATIZED® antibody having variable regions produced by the antibody produced by the hybridoma of American Type Culture Collection (ATCC) Accession No. HB-12119. HB-12119 hybridoma was deposited on May 29, 1996, with the ATCC, currently located at 10801 University Boulevard, Manassas, Va. 20110-2209, under the provision of the Budapest Treaty for the International Recognition of the Deposit of Microorganisms for the Purpose of Patent Procedure (“Budapest Treaty”). Galiximab is a PRIMATIZED® anti-CD80 immunoglobulin (Ig) G1 lambda monoclonal antibody with human constant regions and primate (cynomologous macaque) variable regions. The amino acid and nucleic acid sequences of galiximab and other anti-CD80 antibodies useful in the invention, and methods of obtaining anti-CD80 antibodies, are disclosed in U.S. Pat. No. 6,113,898, which is hereby incorporated by reference in its entirety. Galiximab has been shown to bind CD80 on malignant B cells, and to block CD80-CD28 interaction without interfering with the interaction between CD80 and CD152 (CTLA-4). As described herein, these antibodies may be used to induce apoptosis of HRS cells, to inhibit or reduce HRS cell growth, or to lyse HRS cells or cells of the surrounding infiltrate via ADCC, as herein described.

Additional representative anti-CD80 antibodies or CD80-binding fragments thereof include antibodies and CD80-binding fragments thereof that compete with 16C10 or galiximab for binding to CD80 in a binding inhibition assay. Such binding inhibition assays are well-known to the skilled artisan. The present invention also includes anti-CD80 antibodies and CD80-binding fragments may bind to the same CD80 epitope as 16C10 or galiximab. Anti-CD80 antibodies that compete for binding to CD80 with galiximab and other anti-CD80 antibodies, and that may bind to the same epitope as galiximab are disclosed in U.S. Pat. No. 7,153,508, which is hereby incorporated by reference in its entirety. Methods for determining the binding specificity of antibodies can be determined by immunoprecipitation or by an in vitro assay, such as a radioimmunoassay (RIA) or enzyme-linked immunoabsorbent assay (ELISA) as are well known to the skilled artisan.

The present invention further includes those anti-CD80 antibodies or CD80-binding fragments thereof, which comprise the variable regions the variable regions of the 16C10 antibody, or variable regions derived from 16C10 variable regions, for example, by introduction of one or more amino acid additions, substitutions, or other mutations. Additional anti-CD80 antibodies and CD80 binding fragments useful in the invention include antibodies having residues comprising the antigen binding domain of 16C10, i.e., the complementarity determining regions of 16C10.

Antibodies useful in the invention may be recombinantly prepared using standard techniques. See e.g., Harlow & Lane, Antibodies: A Laboratory Manual, 1988, Cold Spring Harbor Laboratory Press, Cold Spring Harbor, N.Y. and U.S. Pat. Nos. 4,196,265; 4,946,778; 5,091,513; 5,132,405; 5,260,203; 5,658,570; 5,677,427; 5,892,019; 5,985,279; 6,054,561. Representative methods for producing antibodies comprising 16C10 variable regions are described in U.S. Pat. No. 6,113,898. See also U.S. Patent Application Publication Nos. 20030103971 and 20030180290.

Tetravalent antibodies (H₄L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, using heterobifunctional cross-linkers (Wolff et al., Cancer Res., 1993, 53:2560-2565), or by recombinant production to include a dual constant region (Stevenson et al., Anticancer Drug Des., 1989, 3:219-230).

II.B. Small Molecules

The present invention also provides small molecules which may be used as CD80 antagonists. These molecules include heterocyclic compounds of formula (I) as described below and in U.S. patent application Ser. No. 11/539,153. Such compounds inhibit the interaction between CD80 and CD28 and thus the activation of T cells, thereby reducing HRS cells growth as described herein. Examples of heterocyclic compounds that may be employed in the inventive methods include the compounds of formula (I) or a pharmaceutically acceptable salt thereof. These heterocyclic compounds of formula (I) are prepared essentially as described in U.S. Pat. No. 7,081,456. Formula (I) is depicted as follows:

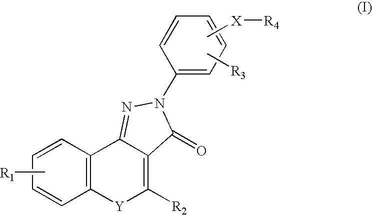

wherein

R₁ and R₃ independently represent H; F; Cl; Br; —NO₂; —CN; C₁ C₆ alkyl optionally substituted by F or Cl; or C₁ C₆ alkoxy optionally substituted by F;

R₂ represents H, or optionally substituted C₁ C₆ alkyl, C₃ C₇ cycloalkyl or optionally substituted phenyl;

Y represents —O—, —S—, N-oxide, or —N(R₅)— wherein R₅ represents H or C₁ C₆ alkyl;

X represents a bond or a divalent C₁ C₆ alkylene radical;

R₄ represents —C(═O)NR₆R₇, wherein R₆ represents a radical of formula -(Alk)_{b}-Q wherein b is 1 and Alk is an optionally substituted divalent straight chain or branched C₁ C₁₂ alkylene, C₂ C₁₂ alkenylene or C₂ C₁₂ alkynylene radical which may be interrupted by one or more non-adjacent —O—, —S— or —N(R₈)— radicals wherein R₈ represents H or C₁ C₄ alkyl, C₃ C₄ alkenyl, C₃ C₄ alkynyl, or C₃ C₆ cycloalkyl, and

Q represents H; —CF₃; —OH; —SH; —NR₈R₈ wherein each R₈ may be the same or different; an ester group; or an optionally substituted phenyl, C₃ C₇ cycloalkyl, C₅ C₇ cycloalkenyl or heterocyclic ring having from 5 to 8 ring atoms; and

R₇ represents H or C₁ C₆ alkyl; or when taken together with the atom or atoms to which they are attached R₆ and R₇ form an optionally substituted heterocyclic ring having from 5 to 8 ring atoms.

Representative molecules of formula (I) include fused pyrazolones having the following structures A and B:

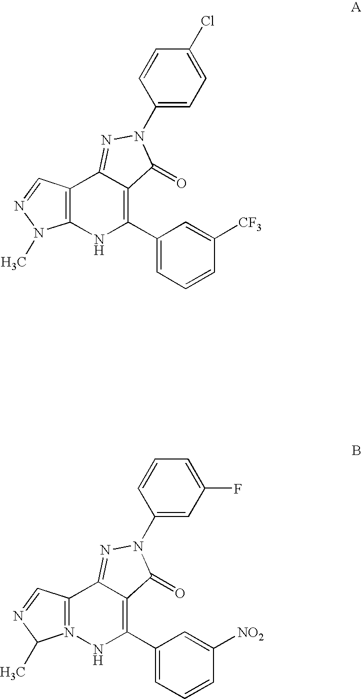

II.C. Activities of HRS Cell Modulators

II.C.1. Induction of Apoptosis or Cell Growth Reduction in HRS Cells

The present invention provides methods for inducing apoptosis or reducing cell growth in a Hodgkins Reed Sternberg cell comprising contacting a CD80 antagonist with the HRS cell or a cell in a surrounding cellular infiltrate, thereby inducing apoptosis or reducing cell growth in the HRS cell. The HRS cells of the present invention may be in vitro, or within a subject, such as a human subject. Reducing cell growth refers to a decrease in the number of cells as a result of a reduction in cell division or in cell size. Inducing apoptosis refers to inducing a highly regulated physiological process of cell death that plays a role in normal development. Cells are continuously exposed to conflicting extracellular signals capable of mediating death (e.g., pro-apoptotic signals) and survival (e.g., anti-apoptotic signals). Disruption of the coordination and/or balance of the molecular mechanisms responsible for regulating these signals is known to be associated with the pathogenesis of Hodgkins lymphoma and other HRS-related diseases.

II.C.1. a. NFκB Pathway

The compositions of the present invention may be used to induce apoptosis or reduce cell growth in HRS cells by inhibiting the NFκB pathway. NFκB pathway inhibition may occur upon contacting the HRS cells or the surrounding cells in the infiltrate with the present HRS modulating compositions.

NFκB is a transcription factor that regulates the expression of various genes involved in apoptosis as well as genes related to cell growth. The NFκB family of proteins, which include p50/p105, p52/p100, p65, RelB and c-Rel, exist as homodimers or heterodimers. In mammalian cells, the dominant responsive form of NFκB is the p50/p65 heterodimer. In almost every cell type, NFκB is present in the cytoplasm in an inactive form and bound to inhibitors including IκBα, IκBβ, and IκBε. Upon activation, IκB is rapidly phosphorylated and ubiquitinated and subsequently degraded by the 26S proteosome. Consequently, NFκB is translocated to the nucleus, where it binds to a specific DNA sequence to induce gene transcription of anti-apoptotic genes including FLICE inhibitory protein (c-FLIP). In HRS cells, NFκB is constitutively expressed.

The constitutive NFκB activation of HRS cells in Hodgkins lymphoma, for example, is essential for tumor-cell survival. Accordingly, the CD80 antagonists of the invention or the CD80 antagonists in combination with agents of the invention as described herein may act to induce apoptosis of HRS cells by diminishing or blocking NFκB activation.

NFκB activation in HRS cells likely results from a variety of mechanisms including receptor-ligand pairing or interaction. The compositions of the invention may act to reduce or block such pairing or interaction, resulting in diminished or inhibited NFκB activation. For example, NFκB may be activated by the pairing of the RANK ligand and RANK receptor, both of which are expressed by HRS cells. Accordingly, the compositions of the invention may diminish or block NFκB activation indirectly by preventing or diminishing the RANK-RANKL ligand-receptor pairing.

Additionally or alternatively, the compositions of the invention may act to diminish or block NFκB activation by reducing or diminishing the cross-linking of CD30 ligand with CD30 receptors. CD30 receptors are expressed on HRS cells and CD30 ligand is expressed by both HRS cells, as well as surrounding cells in the infiltrate. This interaction may result in NFκB activation. Thus, the present compositions may act to diminish or block CD30 ligand-CD30 receptor pairing. Alternatively or additionally, interaction between CD40 ligand and CD40 receptor, a member of the tumor necrosis factor receptor family 1, may also act to activate NFκB. CD40 activates NFκB by promoting turnover of IκBa, an inhibitor of NFκB, as described above. CD40 is expressed on HRS cells, while CD40 ligand is expressed, for example, on the surface of the T lymphocytes in the infiltrate, which surround HRS cells. Thus, the present compositions may act to diminish or block the CD40 ligand-CD40 receptor pairing. NFκB may also be activated in HRS cells via transcriptional control by Notch1. Notch1 belongs to a family of transmembrane receptors that control cell growth, and induce apoptosis of response to extracellular ligands expressed on neighboring cells. During lymphoid development, Notch1 plays a role in the T-cell/B cell lineage decision. Notch1 pathway activation induces translocation of intracellular Notch to the nucleus, where it interacts with the transcription factor CSL. Notch1 is highly expressed on HRS cells. Interaction between intact Notch1 and its ligand, Jagged1, induces proliferation and inhibition of apoptosis. Jagged1 is expressed on HRS cells as well as the cells surrounding HRS cells in the infiltrate. Thus, the compositions of the invention may act to induce apoptosis, or diminish HRS cell proliferation by diminishing or blocking the activation of NFκB by diminishing or blocking the interaction between Notch1 and Jagged1, preventing cells in the infiltrate from signaling or by other mechanisms as described herein.

The compositions of the invention may prevent the pairing or interaction of receptors and ligands as described above in any manner, including by depleting the cells in the surrounding infiltrate by ADCC which express the signaling ligand, by blocking the receptors or by diminishing or preventing the ability of the cells in the surrounding infiltrate from secreting signaling ligands.

Alternatively or additionally, NFκB activation may be induced by the LMP1 receptor expressed on HRS cells. LMP1 shares several features with CD40 and also activates NFκB by promoting turnover of IκBa. However, LMP1 signals independently of a ligand and is dependent on self-association. Thus, the present compositions may act to diminish or block NFκB activation by diminishing the ability of LMP1 to self-associate.

The compositions of the invention may be also used to inhibit, diminish or block NFκB activation, and thereby induce apoptosis, by inhibiting the 26S proteasome. The 26S proteasome is a large protein complex that degrades ubiquitinated proteins and indirectly activates the NFκB pathway. Proteasome inhibition may lead to inactivation of NFκB by inhibiting the degradation of its inhibitor IκB. Specifically, the present compositions may act to inhibit or block the 26S proteasome found in mammalian cells.

The compositions of the invention may be examined for their ability to inhibit the activation of NFκB indirectly by methods well known in the art. For example, the cell-surface expression of 1-CAM-1 on HRS cells may be determined by means of a cell surface fluorescent immuno-binding assay. Because I-CAM-1 is under the regulatory control of NFκB, inhibition of NFκB activation results in reduced levels of these adhesion molecules on the cell surface.

II.C.1. b. Extrinsic Cell Apoptotic Pathway

The compositions of the invention may be used to induce apoptosis of HRS cells by inducing the extrinsic cell death pathway by contacting the HRS cells or the cells in the surrounding infiltrate with the HRS cell modulating compositions as described herein. The extrinsic cell death pathway refers to an apoptotic pathway, which is mediated by the death receptors. Death receptors are cell surface receptors that transmit apoptotic signals initiated by specific death ligands. Death receptors belong to the TNF receptor superfamily, which is defined by the presence of homologous cysteine-rich extracellular domains. There are six members of the TNF receptor superfamily including TNFR1, FAS/CD95, TRAIL-R1, DR5, TRAIL-R2, DR3 (TRAMP), and DR6. All of the death receptors contain an additional cytoplasmic death domain, which enables the receptors to deliver apoptotic signals in sensitive cells upon binding to a specific death ligand. The death ligands include TNF, FasL/CD95L, TRAIL and DR3L. Death ligands bind and oligomerize the death receptors, resulting in a multi-molecular death-inducing signaling complex (DISC). The death domain-signaling molecule, FADD, recruits procaspase-8 molecules to the DISC. The signaling complex results in the activation of caspase-8 via autoproteolytic cleavage, leading to the sequential activation of caspase-3 and caspase-7, followed by destruction of substrates essential for cell survival.

In HRS cells, TNFR1, and TNF-related apoptosis-inducing ligand receptors TRAIL-R1 and TRAIL-R2, as well as CD95L (Fas ligand) are expressed in HRS cells. Nevertheless, HRS cells are often resistant to cell death induction. The HRS cells are likely resistant to cell death due to the constitutive expression of cellular FLICE-inhibitory protein (cFLIP), which acts to inhibit caspase-8. The CD80 antagonists, or the CD80 antagonists in combination with HRS modulating compositions as described below, may be used to induce apoptosis of HRS cells by upregulating pro-apoptotic molecules including caspase molecules such as caspase-3, caspase-8, or the pro-apoptotic molecules CD95 or CD95L. Likewise, the HRS modulating compositions may be used to trigger ubiquitination and degradation of cFLIP. Silencing of cFLIP may act to restore CD95 sensitivity and/or TRAIL signaling. The upregulation of the pro-apoptotic molecules or the downregulation of anti-apoptotic molecules such as cFLIP can be assessed by comparing the amount of these pro-apoptotic or anti-apoptotic molecules or their activity in HRS cells and/or cellular infiltrate treated cells to the amount or activity of these proteins in control HRS cells or control cellular infiltrate cells, i.e., HRS cells or cellular infiltrate cells in the absence of the compositions of the invention. Methods for detecting protein amounts and activities are well known to those of skill in the art.

II.C.1.c. The Intrinsic Apoptotic Pathway

CD80 antagonists, or these compositions in combination with other agents as described herein, may be used to induce the intrinsic apoptotic pathway. The intrinsic apoptotic pathway refers to a pathway controlled by pro-apoptotic proteins including BAX, BAK, BAD, BID, BIM and BMF. The intrinsic pathway of apoptosis triggers the release of these pro-apoptotic proteins from the mitochondria. These pro-apoptotic proteins form a multi-protein complex, the apoptosome, where caspase-9 is activated by dimerization. Caspase-7 is then activated by limited cleavage of caspase-9, leading to the permeabilization of the outer mitrochondrial membrane.

In HRS cells, the intrinsic apoptotic pathway is likely inhibited due to the constitutive expression of the X-linked inhibitor of apoptosis (XIAP) in HRS cells. XIAP binds to caspase-3, caspase-7 and caspase-9 and inhibits the proteolytic activity of these proteins. It is likely that constitutive XIAP expression acts to make HRS cells resistant against apoptosis signals via the intrinsic pathway. Furthermore, the bcl2/BcIXL molecules that are also expressed in HRS cells are known apoptotic antagonists. These molecules act to antagonize the pro-apoptotic function of BAX at the mitochondrial membrane.

Accordingly, the compositions of the invention may induce apoptosis by activating the intrinsic apoptotic pathway, this activation may occur upon contacting the HRS cells or cells in the surrounding infiltrate with the CD80 antagonists or in combination with the other agents described herein. These HRS modulating agents may act to upregulate the pro-apoptotic molecules, such as the caspase molecules, including caspase-7 and caspase-9, and BAX, BAK, BAD, BID, BIM and BMF. Alternatively or additionally, the present compositions may downregulate anti-apoptotic proteins, such as Bcl-x(L) and XIAP. Such upregulation or downregulation can be determined by comparing the amount of pro-apoptotic or anti-apoptotic proteins or the activity of these proteins in HRS treated cells or cells treated in the surrounding cellular infiltrate to the amount or activity of these proteins in control HRS cells or control cells of the surrounding cellular infiltrate according to methods well known to a skilled artisan.

II.C.1 d. STAT Transcription Factors

The compositions of the present invention may also be used to inhibit the growth of HRS cells by inhibiting, diminishing or blocking the activity of the STAT family of transcription factors, which acts to inhibit apoptosis and increase HRS cell proliferation. For example, STATE is constitutively activated in HRS cells. This activation is likely due to the simultaneous expression of IL-13 and IL-13 receptor on HRS cells which results in the activation of STATE via the JAK kinases resulting in an increase in proliferation of HRS cells. Accordingly, the HRS modulating compositions may be used to reduce, diminish or inhibit cell growth in HRS cells by contacting HRS cells or the surrounding cellular infiltrate with the present compositions, thereby diminishing STAT activation. For example, the CD80 antagonists in combination with anti-interleukin-13 antibodies may be used to diminish STAT activation, thereby reducing HRS cell growth. Also, the CD80 antagonists may be used in combination with agents that inhibit IL-13 or IL-13 receptor, such as antibodies directed to the IL-13 receptor, antisense or double-stranded oligonucleotides complementary to IL-13 encoding nucleic acids, or ribozymes that disrupt the IL-13 receptor. See, e.g., U.S. Pat. No. 7,312,024.

II.C.1.e. Diminishing, Inhibiting or Blocking T Cell Activation

The compositions of the present invention may also be used to inhibit the growth of HRS cells by inhibiting, diminishing or blocking T cell activation. As stated above, CD80 is expressed on professional antigen presenting cells and is the natural ligand for CD28 on T cells. Binding of CD80 to CD28 provides a co-stimulatory signal for T cell activation after engagement of the T cells receptor, leading to T cell proliferation and the secretion of cytokines including interleukin-2 (IL-2). HRS cells have been reported to act as antigen expressing cells and to express the IL-2 receptor. Therefore, their ability to induce IL-2 expression from the cells in the surrounding infiltrate, such as from T cells which have been shown to express CD28, (see, e.g., Murray J. Clin. Mol. Pathol. 1995 48:M105-M108) may provide a means by which HRS stimulate their own proliferation. Accordingly, the present CD80 antagonists, such as galiximab, may act to block or reduce the number of T cells which are activated by preventing CD80 from binding to CD28, resulting in the reduction or prevention of cytokine secretion, such as interleukin-2 secretion. Reduced amounts of cytokine secretion may result in the reduction of HRS cell growth.

II.C.2. ADCC

The present invention also provides a method for inducing lysis in a Hodgkins Reed Sternberg cell by antibody-dependent-cellular-cytotoxicity (ADCC) comprising contacting the Hodgkins Reed Sternberg cell with an anti-CD80 antibody or a CD80-binding fragment thereof. Thus, the CD80 antagonists described above, such as the anti-CD80 antibody compositions or the anti-CD80 compositions combined with other antibodies as herein described, may be contacted with HRS cells to attack the HRS cells via ADCC. ADCC refers to a lytic attack on antibody-bound cells, which is triggered following the binding of leukocyte receptors (FcRs) on specialized cells such as natural killer cells, macrophages and neutrophils to the Fc region of the antibody. Thus, the present antibody compositions, such as those comprising antibodies which bind to antigens expressed on HRS cells, may be used to directly kill HRS cells.

Alternatively or additionally, the present invention also provides a method for inducing apoptosis or reducing cell growth in a Hodgkins Reed Sternberg cell comprising contacting a cell in a surrounding cellular infiltrate of a Hodgkins Reed Sternberg cell with an anti-CD80 antibody or a CD80-binding fragment thereof, lysing the cell in the surrounding cellular infiltrate of Hodgkins Reed Sternberg cell by antibody-dependent-cellular cytoxicity, thereby inducing apoptosis or reducing cell growth in the Hodgkins Reed Sternberg cell.

Thus, the CD80 antagonists, such as the anti-CD80 antibody compositions or the anti-CD80 compositions combined with other agents may be contacted with the surrounding cells in which the HRS cells are embedded, resulting in the lytic attack of one or more cell types in the cellular infiltrate. Killing cells in the surrounding infiltrate may act to prevent the HRS cells from receiving anti-apoptotic signals from these cells, which may result in the induction of apoptosis in the HRS cells. For example, endothelial smooth muscle cells, eosinophils and T cells that are recruited by HRS cells engage Notch1, CD30 and CD40 receptors, respectively, on HRS cells contributing to NFκB activation. Thus, by killing these cells via ADCC, NFκB activation may be inhibited, resulting in the induction of apoptosis or reduction in cell growth in HRS cells.

The methods for inducing apoptosis or reducing cell growth in HRS cells as described herein including by ADCC or inhibiting apoptotic pathways, STAT transcription factors, or T cell activation as described above may act to reduce cytokine levels expressed by either the HRS cells or the cells in the surrounding infiltrate. Reduction of cytokine levels may be assessed by determining the amount of cytokine present in the serum of a subject before treatment (i.e., the control level of serum) and compared to the level of cytokine present in the serum of a patient after treatment treated with the present compositions. Methods for determining the level of cytokines in serum are well known to the skilled artisan.

Alternatively or additionally, reduction in cytokine secretion can be determined by comparing the amount of a cytokine secreted by a HRS cell or a cell from the surrounding infiltrate treated with the present compositions, with a control HRS cell or a control cell from the surrounding cellular infiltrate. As stated above, control HRS cells or control cellular infiltrate cells are those cells that have not been treated or contacted with the HRS modulating compositions of the invention.

### III. Therapeutic Applications

The HRS cell modulating compositions including the CD80 antagonists or CD80 antagonists as single agents or in combination with other agents as described herein may also be used to treat HRS-cell related diseases, such as Hodgkins lymphoma, or other diseases characterized by the presence of HRS cells. In one aspect of the invention, methods are provided for treating Hodgkins lymphoma comprising administering to a subject in need thereof a therapeutically effective dose of a pharmaceutical composition comprising a CD80 antagonist, wherein the CD80 antagonist induces apoptosis of a Hodgkins Reed Sternberg cell. For example, the present methods may be used to treat relapsed or refractory Hodgkins lymphoma or any of the types of classical Hodgkins lymphoma.

Classical Hodgkins lymphoma is diagnosed when 1) HRS cells are present 2) the immunophenotype is positive for CD30 and 3) the growth pattern of the disease can be associated with one or more Hodgkins lymphoma types, as described below. Refractory Hodgkins lymphoma refers to a Hodgkins lymphoma that has not responded to an initial therapy. Relapsed Hodgkins lymphoma refers to a Hodgkins lymphoma that initially responds to therapy, but does not respond to subsequent treatment. The types of Hodgkins lymphoma include Nodular Sclerosis Hodgkins lymphoma, Lymphocyte Predominant Hodgkins lymphoma, Mixed Cellularity Hodgkins lymphoma and Lymphocyte Depleted Hodgkins lymphoma.

Nodular Sclerosis Hodgkins lymphoma is characterized by lymph nodes in the lower neck, chest and collarbone that usually contain normal and reactive lymphocytes as well as HRS cells. The HRS cells are separated by bands of scar-like tissues and the predominant HRS morphology is of the lacunar cell, which is characterized by a lobulated nucleus, prominent eosinophilic nucleolus and abundant clear cytoplasm. Nodular sclerosis accounts for 60-70% of Hodgkins cases and appears to account for the increase in Hodgkins cases in recent years. In contrast, Lymphocyte Predominant Hodgkins lymphoma has a lymphocyte-rich cellular background containing typical HRS cells. Lymphocyte Predominant Hodgkins lymphoma accounts for 5% of Hodgkins cases and affects more men than women. Lymphocyte Depleted Hodgkins lymphoma is also characterized by HRS cells, but these cells display unusual cytological features. Additionally, Lymphocyte Depleted Hodgkins lymphoma may be characterized by few HRS cells and lymphocytes with scar-like tissue. In Mixed Cellularity Hodgkins lymphoma, the lymph nodes also contain HRS cells and inflammatory cells. This lymphoma type accounts for 20-30% of Hodgkins cases.

The present compositions may be used to treat Hodgkins lymphoma by administering to a subject in need thereof a therapeutically effective dose of a anti-CD80 antagonist. A therapeutically effective dose refers to an amount of an HRS cell modulating composition sufficient to result in amelioration of symptoms of disease. For the treatment of Hodgkins lymphoma, therapeutic effects are measured as known by one of skilled in the art using measurable clinical outcomes such as reduction in tumor mass and/or the number of nodules related to lymphoma, reduction of abnormally large spleen or liver, reduction of neoplastic B cells in bone marrow, inhibition of or slowed lymphoma cell growth, reduction or disappearance of metastases, and progression-free survival.

Additional indices of therapeutic effect include reduction of the number of HRS cells, induction of apoptosis or ADCC-mediated lysis of HRS cells, reduced or slowed growth of HRS cells, downregulation of NFκB activation in HRS cells, upregulation of the activity or expression of a pro-apoptotic molecule in HRS cells, downregulation of the activity or expression of an anti-apoptotic molecule in HRS cells, and/or reduction of cytokine levels secreted by HRS cells or in the cellular infiltrate surrounding HRS cells. When assessing a change in any of the above-noted measurements, the change is assessed relative to a control level or sample, for example a level of an indices as observed in a subject prior to administration of a CD80 antagonist, or a level of an indices as observed in a HRS cell or cells of the surrounding infiltrate not contacted with a CD80 antagonist. For example, a change in any of the above-noted indices may be a change of at least about two-fold greater or less than a control level, or at least about five-fold greater or less than a control level, or at least about ten-fold greater or less than a control level, at least about twenty-fold greater or less than a control level, at least about fifty-fold greater or less than a control level, or at least about one hundred-fold greater or less than a control level. A change in any of the above-noted indices may also be a change of at least 10% greater or less than a control level, or at least 20% greater or less than a control level, or at least 30% greater or less than a control level, or at least 40% greater or less than a control level, or at least 50% greater or less than a control level, or at least 60% greater or less than a control level, or at least 70% greater or less than a control level, or at least 80% greater or less than a control level, or at least 90% greater or less than a control level, or at least 100%, or more greater or less than a control level.

Toxicity and therapeutic efficacy of the present compositions can be determined by standard pharmaceutical procedures in cell cultures or experimental animals. While compositions which exhibit toxic side effects may be used, care should be taken to design a delivery system that targets such compositions to the site of affected tissue in order to minimize potential damage to uninfected cells and, thereby, reduce side effects.

The data obtained from the cell-based assays and animal studies, usually in rodents, rabbits, dogs, pigs, and/or or primates, can be used in formulating a range of dosage for use in humans. The animal model may also be used to determine the appropriate concentration range and route of administration. Such information may then be used to determine useful doses and routes for administration in humans. Typically, a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine. Efficacious doses of anti-CD80 antibody, such as galiximab, may include weekly doses ranging from 100 mg/m² to 600 mg/m², such as 4 weekly infusions of galiximab at a dose of 125 mg/m², 250 mg/m², 375 mg/m², or 500 mg/m². Anti-CD80 antibodies may also be administered at lower or higher doses, such as doses that are about 90%, or about 80%, or about 70%, or about 60%, or about 50%, or about 40%, or about 30%, or about 20% or about 10%, or less of the above-identified doses for galiximab. For example, effective doses may include weekly doses of 10 mg/m², 12.5 mg/m², 25 mg/m², 37.5 mg/m², or 50 mg/m². In particular, non-fucosylated anti-CD80 antibodies having enhanced ADCC activity, or other enhanced anti-tumor activity, when compared to the same antibody, which is prepared in a manner that does not specifically remove fucose residues, may be effective at a reduced dose. Doses of anti-CD80 antibodies may also include doses that are about 110%, or about 120%, or about 130%, or about 140%, or about 150%, or about 160%, or about 170%, or about 180% or about 190%, or more of the above-identified doses for galiximab.

The dosage of such compounds lies preferably within a range of circulating concentrations with little or no toxicity. The dosage may vary within this range depending upon the dosage form employed and the route of administration utilized. For any HRS cell modulator used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in a conventional manner using one or more physiologically acceptable carriers or excipients. Thus, the compositions and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, rectal administration, or within/on implants, e.g., matrices such as collagen fibers or protein polymers, via cell bombardment, in osmotic pumps, grafts comprising appropriately transformed cells, etc.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Pharmaceutical compositions may also include various buffers (e.g., Tris, acetate, phosphate), solubilizers (e.g., TWEEN®, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as TWEEN®-20, TWEEN®-80, NP-40 or TRITON-X®-100. EBP may also be incorporated into particulate preparations of polymeric compounds for controlled delivery to a patient over an extended period of time. A more extensive survey of components in pharmaceutical compositions is found in Remington's Pharmaceutical Sciences, 1990, 18th ed., A. R. Gennaro, ed., Mack Publishing, Easton, Pa.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

For combination therapies, which include a CD80 antagonist and one or more additional therapeutic agents, such as antibodies, small molecules or peptides as herein described are administered within any time frame suitable for performance of the intended therapy. Thus, the single agent CD80 antagonist such as an anti-CD80 antibody or anti-CD80-binding fragment thereof and any of the additional agents as described herein may be administered substantially simultaneously (i.e., as a single formulation or within minutes or hours) or consecutively in any order. For example, an anti-CD80 antibody compositions may be administered within about 1 year of from e.g., a second antibody, such as anti-interleukin-13 antibody within about 10, 8, 6, 4, or 2 months, or within 4, 3, 2 or 1 week(s), or within about 5, 4, 3, 2 or 1 day(s). The administration of the anti-CD80 antibody and a second therapeutic HRS modulator preferably elicits a greater reduction in HRS cells or tumors dependent on HRS cells for growth than administration of either alone.

For additional guidance regarding formulation, dose, administration regimen, and measurable therapeutic outcomes, see Berkow et al., The Merck Manual of Medical Information, 2000, Merck & Co., Inc., Whitehouse Station, New Jersey; Ebadi, CRC Desk Reference of Clinical Pharmacology, 1998, CRC Press, Boca Raton, Fla.; Gennaro, Remington: The Science and Practice of Pharmacy, 2000, Lippincott, Williams & Wilkins, Philadelphia, Pa.; Katzung, Basic & Clinical Pharmacology, 2001, Lange Medical Books/McGraw-Hill Medical Pub. Div., New York; Hardman et al., Goodman & Gilman's the Pharmacological Basis of Therapeutics, 2001, The McGraw-Hill Companies, Columbus, Ohio; Speight & Holford, Avery's Drug Treatment: A Guide to the Properties, Choices, Therapeutic Use and Economic Value of Drugs in Disease Management, 1997, Lippincott, Williams, & Wilkins, Philadelphia, Pa.

### IV. Combination Therapies

The CD80 antagonists of the present invention may be used in combination with other HRS modulating compounds. When used in combination with one or more additional therapeutic agents, the CD80 antagonist and the one or more additional agents may be administered or otherwise contacted with cells concurrently of sequentially in either order. The disclosed combination therapies may elicit a synergistic therapeutic effect, i.e., an effect greater than the sum of their individual effects. Measurable therapeutic effects are described herein above. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

Specifically, the invention provides a method of treating Hodgkins lymphoma comprising administering to a subject in need thereof a therapeutically effective dose of a CD80 antagonist and an antibody selected from the group consisting of an anti-CD30 antibody, a CD30 binding fragment of the anti-CD30 antibody, an anti-CD40 antibody, a CD40-binding fragment of the anti-CD40 antibody, an anti-RANK antibody, a RANK-binding fragment of the anti-RANK antibody, an anti-RANKL antibody, a RANKL-binding fragment of the anti-RANKL antibody, an anti-TRAIL antibody, a TRAIL-binding fragment of the anti-TRAIL antibody, an anti-Notch antibody, a Notch-binding fragment of the anti-Notch antibody, an anti-LMP antibody, a LMP-binding fragment of the anti-LMP antibody, an anti-IL-13 antibody, a IL-13-binding fragment of the anti-IL-13 antibody, an anti-CD20 antibody, a CD20-binding fragment of the anti-CD20 antibody, an anti-CD52 antibody, and a CD52-binding fragment of the anti-CD52 antibody, CD80 antagonists may also be used in combination with one or more additional agents that deplete regulatory T cells, for example, an anti-CCR4 antibody, or which otherwise block, inhibit, or downregulate regulatory T cell functions to thereby elicit synergistic therapeutic effects.

Anti-CD30 antibodies specifically bind to CD30 receptors, which are densely expressed on HRS cells, and rarely expressed in normal cells. In addition to its transmembrane form, CD30 is also shed in a soluble form, which can be detected in the sera of patients with Hodgkins lymphoma. Anti-CD30 antibodies are known in the art and include the fully humanized native antibody MDX-060 (Mederex), and the chimeric antibody SGN-30 (Seattle Genetics).

Anti-CD40 antibodies specifically bind to CD40 receptors which are expressed on HRS cells, as well as monocytes, dendritic cells and B cells. Anti-CD40 antibodies are known in the art and include SGN-40 (e.g., Advanti et al. (2005) Blood, 106:1504).

Anti-RANK and anti-RANKL antibodies may also be used in combination with anti-CD80 antibodies of the present invention. Anti-RANK antibodies specifically bind to RANK, a transmembrane protein. In healthy individuals, RANK receptor expression is restricted to dendritic cell, T cells and osteoclast precursor cells. RANK receptor, when paired with its ligand RANKL, may be necessary for bone metabolism, lymph node formation, B cell development and dendritic cell survival. HRS cells aberrantly express RANK and RANKL, creating an autocrine loop that is involved in regulating cytokine secretion and HRS cell survival. Anti-RANK antibodies can be derived according to methods well known in the art and as described herein. Anti-RANKL antibodies are also known in the art and include AMG162 (Amgen, Inc).

Anti-TRAIL antibodies may also be used in combination with the anti-CD80 antibodies of the present invention. Anti-TRAIL antibodies specifically bind to TRAIL receptors. The TRAIL receptor ligand, a death protein, has four receptors, including TRAIL-R1, TRAIL-R2, TRAIL-R3 and TRAIL-R4. TRAIL-R1 and TRIAL-R2 are death receptors that can initiate caspase activation. HRS cells express TRAIL receptors R1, R2 and R4 and anti-TRAIL receptor antibodies may be used to induce ADCC in HRS cells as described herein.

Anti-IL13 antibodies may also be used in combination with the anti-CD80 antibodies of the present invention. Anti-IL13 antibodies specifically bind to interleukin-13. Both interleukin-13 and interleukin receptor are overexpressed in HRS cells. IL-13 and IL-13Rα-1 are believed to enhance the survival of HRS cells by an IL-13 autocrine and paracrine cytokine loop. Anti-IL13 antibodies are known in the art and include CAT-354 (Cambridge Antibody Technology).

Anti-Notch1 antibody may also be used in the present compositions. Anti-Notch1 antibodies specifically bind to Notch1 which is expressed on HRS cells. Activation of Notch1 leads to NFκB activation which inhibits apoptosis of HRS cells. Anti-Notch1 antibodies are known in the art and can be manufactured according to well known methods as described herein.

Anti-LMP antibodies bind to latent membrane proteins 1 and 2, which are expressed on HRS cells in Epstein Barr virus positive cases and may be responsible for NFκB activation. Anti-LMP antibodies are known in the art and can be manufactured according to well known methods as described herein.

In addition to the above-described antibodies, antibodies which bind to antigens expressed by cells in the surrounding infiltrate and not by HRS cells may also be combined with the CD80 antagonists of the present invention. Such antibodies may aid in the eventual induction of apoptosis or death of HRS cells by lysing the anti-apoptotic secreting cells in the surrounding cellular infiltrate. Such antibodies include anti-CD20 antibodies and anti-CD52 antibodies.

Anti-CD20 antibodies specifically bind to CD20 which is infrequently expressed by HRS cells. However, CD20 is expressed on B cells which are present in the surrounding cellular infiltrate. Anti-CD20 antibodies are well known in the art and include RITUXAN®.

Anti-CD52 antibodies may also be combined with anti-CD80 antibodies of the present invention. Anti-CD52 antibodies specifically bind to CD52 which is expressed on normal and neoplastic B and T lymphocytes, monocytes and natural killer cells. Many of these CD52 expressing cells can be found in the cellular infiltrate surrounding HRS cells. Anti-CD52 antibodies are well known in the art and include Alemtuzumab (CAMPATH®).

The antibodies described above, which may be used in combination with the CD80 antagonists such as the anti-CD80 antibodies of the present invention, may include polyclonal antibodies, monoclonal antibodies (mAbs), humanized or chimeric antibodies, PRIMATIZED® antibodies, which contain human constant regions and primate (cynomolgus macaque) variable regions, human monoclonal antibodies, single chain antibodies, Fab fragments, F(ab′)₂ fragments, fragments produced by a Fab expression library, anti-idiotypic (anti-Id) antibodies, Fc fusion proteins, and epitope-binding fragments of any of the above.

Additional agents that may be used in combination with CD80 antagonists include agents that block interaction of B lymphocyte stimulator (BLyS) with one or more of its receptors, B cell activating factor receptor (BAFF-R), transmembrane activator and calcium modulator and cyclophilin ligand interactor (TACI), or B-cell maturation antibody (BCMA). For example, useful agents include antibodies that specifically bind the BLyS ligand or antibodies that specifically bind one or more of its receptors, including any of the antibody types described herein. Small molecule inhibitors of the interaction of BLyS with one or more of its receptors may also be used.

The CD80 antagonists of the present invention may also be combined with small molecules to induce apoptosis or reduce cell growth in HRS cells. Specifically, the invention provides a method of treating Hodgkins lymphoma comprising administering to a subject in need thereof a therapeutically effective dose of a pharmaceutical composition comprising a CD80 antagonist and a proteosome inhibitor or a histone deacytylase inhibitor, wherein the composition induces apoptosis or reduces cell growth in HRS cells. Such small molecules include, proteosome inhibitors such as Bortezomib (PS-341; Millennium Pharmaceuticals) and MG-132 (Tokyo, Metroplitan Institute of Medical Science). Other small molecules for use in combination with the antibody compositions described herein include histone deacytylase inhibitors, which may act to inhibit the anti-apoptotic HRS cell pathways as described herein. Examples of histone deacytlyase inhibitors include depsipeptide (FK228; Gloucester Pharmaceuticals) and suberoylanilide hydroxamic acid (SAHA; Aton Pharma). Other small molecules that may be used to induce apoptosis of HRS cells in combination with the present CD80 antagonists include triterpenoids, such as CDD (RTA401, Reata Discovery) and 17-allylamino-17-demethoxy-gledanamycin (see, e.g., Kamal et al., 2004, Trends. Mol. Med. 10, 283-290).

The CD80 antagonists of the present invention may also be combined with peptides to induce apoptosis of HRS cells. Such peptides include a proteosome inhibitor, such as peptide aldehydes, e.g., N-acetyl-leucinyl-leucynil-norleucynal, N-acetyl-leucinyl leucynil-methional, carbobenzoxyl-leucinyl-leucynil-norvalinal, carbobenzoxyl-leucinyl-leucynil-leucynal, lactacystine, b-lactone, boronic acid peptides, ubiquitin ligase inhibitors, cyclosporin A, and deoxyspergualin.

Additional representative agents useful for combination therapy include cytotoxins, radioisotopes, chemotherapeutic agents, immunomodulatory or immunoregulatory agents, anti-angiogenic agents, anti-proliferative agents, pro-apoptotic agents, and cytostatic and cytolytic enzymes (e.g., RNAses). Additional agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. CD80 antagonists of the invention may also be used in combination with agents that deplete regulatory T cells, or which block, inhibit, or otherwise downregulate regulatory T cell functions.

The term cytotoxin generally refers to an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine, methotrexate, fluorouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Radioisotopes suitable for radiotherapy include but are not limited to α-emitters, β-emitters, and auger electrons. These radioisotopes are typically conjugated to a targeting antibody for delivery to disease cells. For example, radiolabeled antibodies can include a radioisotope such as ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ¹²¹mtellurium, ⁹⁹technetium, ^{122m}tellurium, ^{125m}tellurium, ¹⁶⁵thulium, ¹⁶⁷thulium, ¹⁶⁸thulium, ⁹⁰yttrium, alpha emitters, such as ²¹³bismuth, ²¹³lead, and ²²⁵actinium, and nitride or oxide forms derived there from.

Immunomodulatory or immunoregulatory agents are compositions that elicit an immune response, including humoral immune responses (e.g. production of antigen-specific antibodies) and cell-mediated immune responses (e.g. lymphocyte proliferation). Representative immunomodulatory agents include cytokines, xanthines, interleukins, interferons, and growth factors (e.g., TNF, CSF, GM-CSF and G-CSF), and hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Immunomodulatory agents useful in the invention also include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

Additional drugs useful in the invention include anti-angiogenic agents that inhibit blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3-VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

CD80 antagonists of the invention may also be used in combination with anti-cancer therapeutic antibodies and antibody/drug conjugates. Representative antibodies, which may be used in unlabeled/unconjugated form or as an antibody/drug conjugate, include anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-CD33 antibody/drug conjugates, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

CD80 antagonists of the invention may also be used in combination with systemic anti-cancer drugs, such as epithilones (BMS-247550, Epo-906), reformulations of taxanes (Abraxane, Xyotax), microtubulin inhibitors (MST-997, TTI-237), hsp90 inhibitors (geldanamycin and derivatives thereof, such as 17-allylamino-17-demethoxygeldanamycin, SNX-5422 (Serenex), STA-9090 (Synta Pharmaceuticals), CCT0180159 (The Institute of Cancer Research), inhibitors of DAX-1, inhibitors of mammalian target of rapamycin/mTOR(CCI-779, AP23573 and RAD-001), etc.

In other combination therapies, CD80 antagonists may be administered together with one or more combinations of chemotherapeutic agents, for example, alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chiorambucil, chlornaphazine, cholophosphamide, estramustine, ifosfamide, mechiorethamine, mechiorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofuran; spirogermanium; tenuazonic acid; triaziquone; 2, 2′, 2′-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology of Princeton, New Jersey) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperarnicins; capecitabine and combinations of therapeutic agents such as ABVD (adriamycin, bleomycin, vincristine, dacarbazine).

Throughout this application, various publications, patents and published patent applications are referred to by an identifying citation. The disclosures of these publications, patents and published patent specifications referenced in this application are hereby incorporated by reference into the present disclosure to more fully describe the state of the art to which this invention pertains.

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention.

### EXAMPLES

### Example 1

### CD80 Expression on Hodgkins Lymphoma Cell Lines

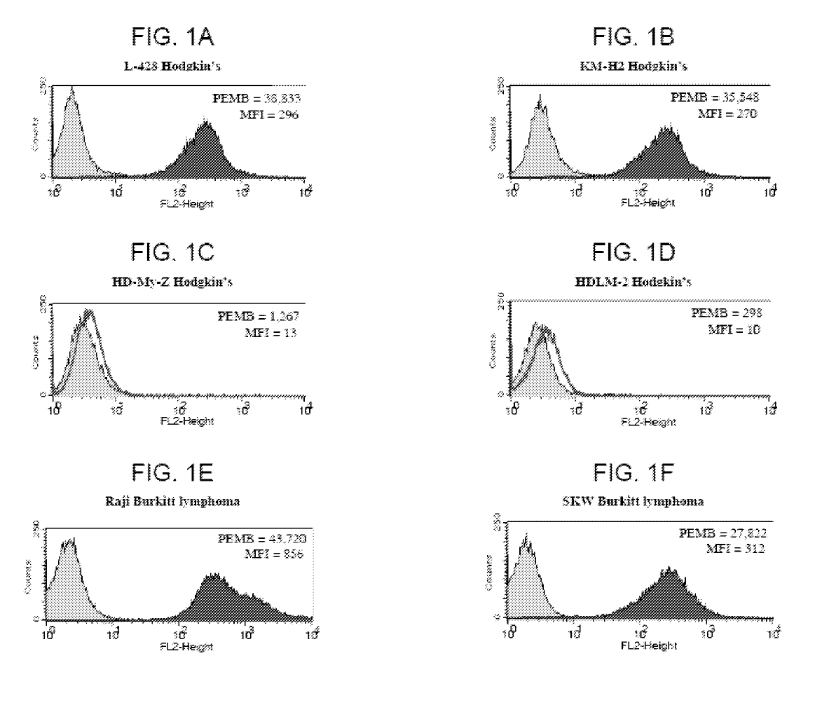

One million cells were recovered from cultures of various Hodgkins lymphoma cell lines, including L-428, KM-H2, HD-My-Z, and HDLM-2 cell lines. Raji Burkitt lymphoma cells and SKW Bukitt's lymphoma cells, which are known to express CD80, were used as controls. The cells were stained for 1 hour on ice with phycoerythrin (PE)-labeled anti-human CD80 antibody (Clone L307.4, available from BD Biosciences of San Jose, Calif.). Propidium iodide was added to samples and FACS expression was determined using live cell gating. For calculation of relative receptor expression, the number of PE molecules bound to the cells (PEMB) was estimated using PE QUANTIBRITE™ beads (BD Biosciences). The results are shown in FIGS. 1A-1F.

In a separate experiment, one million L-428 Hodgkin lymphoma cells were recovered from culture and incubated with varying concentrations of galiximab (0.05 μg/ml, 0.5 μg/ml, 5 μg/ml, and 50 μg/ml) for one hour on ice. Cells were washed and bound antibody was detected with 2.5 μg/ml of goat anti-human F(ab′)₂ IgG-FITC (Southern Biotechnology Associates of Birmingham, Ala.) for one hour on ice. Unbound antibody was washed off, propidium iodide was added to the cells, and cells were analyzed using live cell gating. Galiximab showed concentration dependent binding to L-428 Hodgkin lymphoma cells.

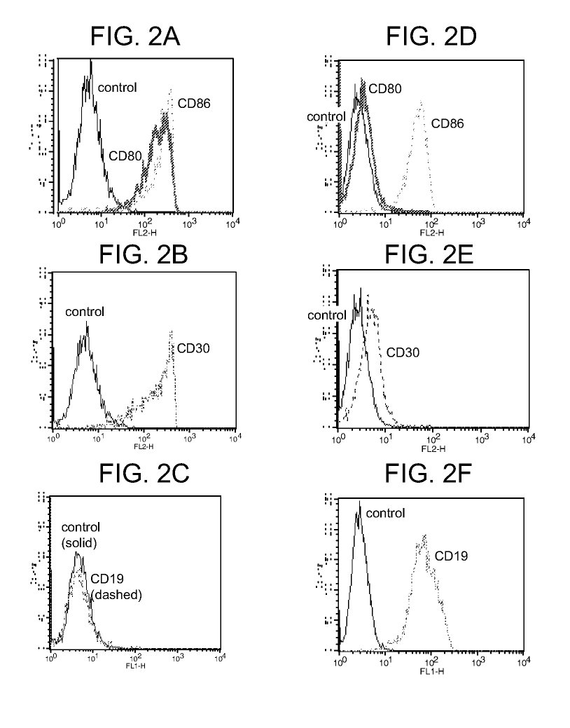

The L-428 Hodgkin lymphoma cell line was further analyzed by flow cytometry for expression of CD80, CD86, CD30, and CD19 using standard methods. Antibodies used for flow cytometric analysis include L307.4 (anti-CD80; BD Biosciences of San Jose, Calif.), IT2.2 (anti-CD86; eBiosciences of San Diego, Calif.), BerH8 (anti-CD30; BD Biosciences of San Jose, Calif.), and HIB19 (anti-CD19; BD Biosciences of San Jose, Calif.). Isotype matched control antibodies were analyzed concurrently. The L-428 cell line exhibited strong expression of the Hodgkins lymphoma marker CD30 and did not express the mature B cell marker CD19 (FIGS. 2B-2C) For comparison, the non-Hodgkins lymphoma cell line Ramos exhibited strong expression of CD19 and low level expression of CD30 (FIGS. 2E-2F). The L428 Hodgkins lymphoma cell line also demonstrated high levels of expression of both CD80 and CD86 (FIG. 2A), whereas, the Ramos non-Hodgkins lymphoma cell line exhibited strong expression of CD86 and low level expression of CD80 (FIG. 2D).

### Example 2

### ADCC Activity of Galiximab on Hodgkins Lymphoma Cells

The ability of the galiximab to induce ADCC of L-428 Hodgkins lymphoma cells was tested in a cytotoxicity chromium release assay. Human effector cells were prepared from whole blood from three donors. Briefly, human peripheral blood mononuclear cells were purified from heparinized whole blood by standard Ficoll-paque separation. The cells were resuspended in GIBCO™ RPMI1640 media containing 10% FBS and 200 U/ml of human IL-2 and incubated overnight at 37° C. The following day, the cells were collected and washed once in culture media and resuspended at 1×10⁷ cells/ml. Target cells were incubated with 100 μCi ⁵¹Cr for 1 hour at 37° C. The target cells were washed once to remove the unincorporated ⁵¹Cr, and plated at a volume of 1×10⁴ cells/well. Target cells were incubated with 50 μl of effector cells and 50 μl of antibody. An anti-CD30 antibody (murine IgG2b) and an anti-RF2 antibody (human IgG1) were used as negative controls. Following a four hour incubation at 37° C., the supernatants were collected and counted on a gamma Counter (Isodata Gamma Counter, Packard Instruments of Meriden, Connecticut). The counts per minute were plotted as a function of antibody concentration. The percentage lysis of target cells was calculated as follows:

$$\% Lysis = \frac{sample Release ( CPM ) - spontaneous release ( CPM )}{maximum release ( CPM ) - spontaneous CPM )} \times 100 \%$$

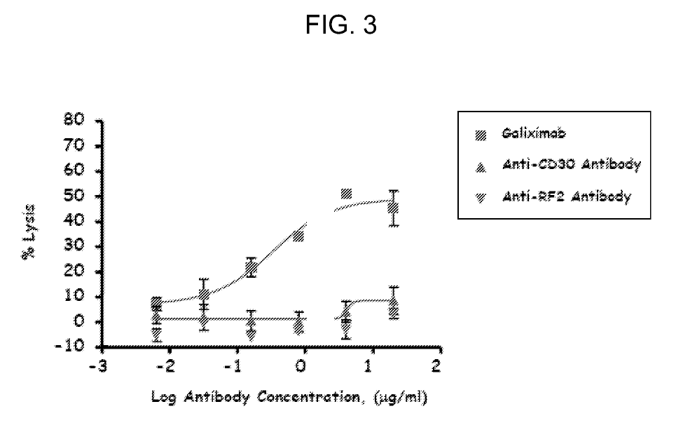

As shown in FIG. 3, galiximab effectively induced ADCC of L-428 Hodgkins lymphoma cells.

### Example 3

### Galiximab for Treatment of Relapsed or Refractory Hodgkins Lymphoma

Adult patients (at least 18 years old) with histologically confirmed classical Hodgkins lymphoma who have relapsed or failed to respond to at least one prior standard chemotherapy are eligible for treatment with the anti-CD80 antibody, galiximab. Galiximab is an IgG1 lambda, anti-CD80 monoclonal antibody developed using PRIMATIZED® antibody technology to decrease immunogenicity. The variable regions are of cynomologous macaque origin, and the constant regions are of human origin. Galiximab is formulated for intravenous injection as a sterile product in 10 mmol/L sodium citrate, containing 150 mmol/L sodium chloride and 0.02% polysorbate 80 at pH 6.5.

For the purposes of a clinical trial, patients tested to assess efficacy of galiximab have not been previously treated with galiximab, radiotherapy, biologic therapy, chemotherapy, or immunosuppressive therapy within 3 weeks before the first scheduled galiximab treatment. Inclusion criteria encompasses patients with a measurable disease with at least one lesion≧10 mm; and adequate hematologic, renal and hepatic function. For the purposes of the clinical trial, patients are excluded if they have CNS lymphoma, active opportunistic infection, or serious non-malignant disease, such as uncontrolled diabetes mellitus or are HIV positive. No concomitant therapy with additional lymphoma treatments or other investigational drugs is performed.

Patients are treated with galiximab for a period of one month. The patients are pre-medicated with 50 mg of diphenhydramine administered by intravenous infusion (IV) using an infusion pump and a 0.22 micron low-protein binding filter or by mouth and 650 mg of orally administered acetaminophen prior to intravenous infusions of galiximab. Patients receive 500 mg/m² of galiximab once weekly for 4 weeks. Galiximab is administered in an outpatient setting over a 1-hour period. Patients are monitored for at least one hour following completion of galiximab infusion. Antibody doses are diluted in 150 mL-250 mL of normal saline. Galiximab infusions are withheld if any infusion reaction (e.g., fever, chills, dyspnea)≧grade 1 occurs. After resolution of the symptoms, the infusion may be restarted at half the previous rate. Galiximab infusion is stopped if the patient experiences a second infusion-related adverse event, and not restarted the same day. No dose modifications are made except for discontinuation secondary to life-threatening toxicity.

After the administration of the induction therapy as described above, patients receive extended galiximab therapy on a monthly basis until disease progression. Patients receive 500 mg/m² doses of galiximab every four weeks over 60 minutes using an infusion pump and a 0.22 micron low-protein binding filter. The infusion time is extended if the patient experiences infusion-related toxicity. Patients who respond to galiximab are allowed to continue treatment. Their progress is examined at week eight and then after every 3 galiximab treatments (i.e., every 12 weeks) until disease progression. All patients who withdraw from the study early or who are alive at the end of the 48-month study period are observed at 6-month intervals for continuation of response, initiation of other lymphoma therapy, survival status or cause of death.

Evaluation of disease is performed by comprehensive scans (computed tomography, magnetic resonance imaging, and x-rays) and physical examination at baseline (study entry), 1 month after completion of galiximab treatment (day 50), every 3 months thereafter for years 1 and 2, and every 6 months for years 3 and 4. Response to treatment is analyzed using standard outcome measures for clinical trials (complete response, unconfirmed complete response, partial response, stable disease and progressive disease) as defined by the International Workshop Response Criteria for Non-Hodgkins lymphoma. Relevant end points include overall response rate, complete remission rate, unconfirmed complete remission rate, partial remission rate, duration of response and time to progression.

### Example 4

### Pharmacokinetic Analysis of Galiximab

Serum samples are obtained before infusion, and within 10 minutes of the completion of infusions (days 1, 8, 15 and 22), prior to galiximab infusion at week eight and thereafter every 12 weeks for as long as the patient remains on the study. Samples at week eight are only collected prior to galiximab infusion; a post infusion pharmacokinetic analysis sample is not needed at these time points. Five mL of venous blood are collected at each time point in a red top VACUTAINER® tube. Serum is separated from the blood cells within one hour by centrifugation, removed, and stored in polypropylene transport tubes at −20° C. Blood cells are discarded. If more than one hour elapses before centrifugation, whole blood samples are refrigerated at 2-8° C.

Pharmacokinetic analysis analyses include galiximab serum concentrations, maximum observed concentration (Cmax), time to maximum observed concentration, serum half-life, and the area under the concentration time curve (AUC).

Data are analyzed using a non-compartmental linear regression method to determine the serum half-life using data from all samples collected after study day 1 that contain galiximab concentrations exceeding the lower limit of quantitation for the assay (250 ng/mL). AUC is calculated using the linear/logarithmic trapezoidal method and determined with time extrapolated out to infinity.

### Example 5

### Effect of Galiximab on Cytokine Levels

As described herein, growth of Hodgkins lymphoma depends in part on the immune microenvironment of HRS cells, including cytokine secretion by the surrounding cellular infiltrate which includes, T cells, B cells, and dendritic cells. The immuno-modulatory anti-CD80 monoclonal antibody, galiximab, may exert its effects from direct cytotoxicity on tumor cells or from regulation of the immune microenvironment. HRS cells produce a large number of cytokines (e.g., IL-1, II-6, IL-9, IL-13) and respond to a large number of cytokines produced by surrounding immune cells (e.g., IL1α, ILβ, IL2, IL6, IL10, tumor necrosis factor and lymphotoxin A).

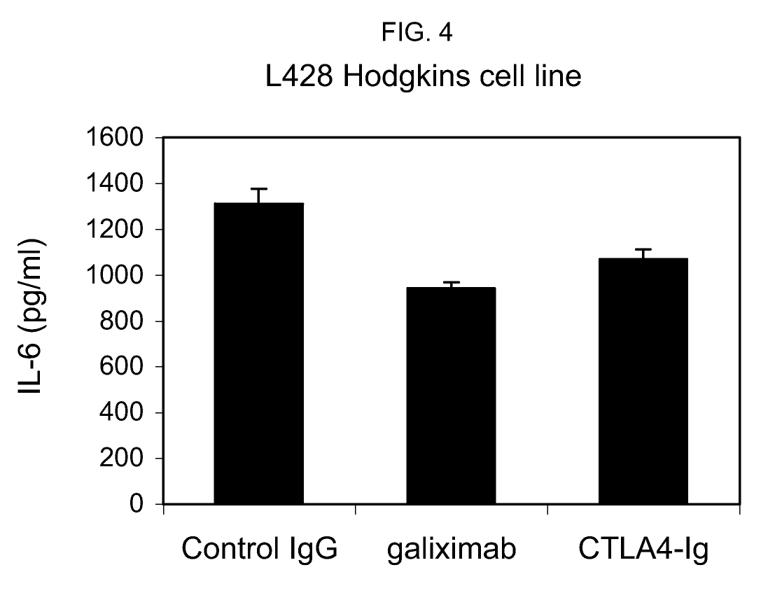

The effect of galiximab on the expression of IL-6 by the L428 Hodgkins lymphoma cell line was investigated. Briefly, L-428 cells were cultured at 4×10⁴ cells per well in a 96-well, flat-bottom tissue culture microtiter plate in RPMI-1640 media containing 10% heat-inactivated fetal bovine serum. Culture supernatants were harvested, in duplicate, after 4 days and a multiplexed cytometric bead array (BD Biosciences of San Jose, Calif.) was used to measure the concentration of IL-6 present in culture supernatants. Cells were cultured in the presence of galiximab (10 μg/ml), purified human serum myeloma IgG1, kappa (10 μg/ml) (Southern Biotech of Birmingham, Ala.) as an isotype control for galiximab, and CTLA4-Ig (10 μg/ml) purified from a engineered CHO production cell line as a control to compare a combined CD80/CD86 blockade with specific blockade of CD80 mediated by galiximab. As shown in FIG. 4, galiximab partially inhibited the expression of IL-6 by the L-428 Hodgkins lymphoma cell line. Also, L-428 cells express similar levels of CD80 and CD86 (FIG. 2A), but galiximab inhibited interleukin-6 expression to a level similar to or greater than the inhibition mediated by CTLA4-Ig.

The effect of galiximab on cytokine production is also assessed in patients. Serum is collected at the following time points: prior to treatment on day 1, in week 4 (completion of induction), in week 8, and every 12 weeks thereafter for the duration that the patient remains on study. All samples for tests of immune function are drawn prior to galiximab infusion. Five mL of venous blood are collected in a red top VACUTAINER® tube. Serum is separated from the blood cells within one hour by centrifugation, removed, and stored in polypropylene transport tubes at −20° C. Blood cells are discarded. If more than one hour elapses before centrifugation, whole blood samples are refrigerated at 2-8° C. Changes in serum levels of cytokines are correlated with treatment response.

### Example 6

### Galiximab and Rituximab for Treatment of Relapsed or Refractory Hodgkins Lymphoma

Patients with relapsed or refractory Hodgkins disease are treated with galiximab as described above and rituximab (RITUXAN®). Patients are treated with 375 mg/m² of rituximab and 500 mg/m² of galiximab on a dosing schedule as described above for single agent galiximab treatment. Therapeutic efficacy is assessed using indices as described in Examples 3 and 5.

### Example 7

### Galiximab and Bortezomib for Treatment of Relapsed or Refractory Hodgkins Lymphoma

Patients with relapsed or refractory Hodgkins disease are treated with galiximab as described above and bortezomib (Millennium Pharmaceuticals). Patients are treated with 1.3 mg/m² of bortezomib and 500 mg/m² of galiximab on a dosing schedule as described above for single agent galiximab treatment. Therapeutic efficacy is assessed using indices as described in Examples 3 and 5.

### Example 8

### Galiximab and Suberoylanilide Hydroxamic Acid for Treatment of Relapsed or Refractory Hodgkins Lymphoma

Patients with relapsed or refractory Hodgkins disease are treated with galiximab as described above and suberoylanilide hydroxamic acid (SAHA). Patients are treated orally with 400 mg orally once daily for the cycle time described above for galiximab. Therapeutic efficacy is assessed using indices as described in Examples 3 and 5.

1. A method of treating Hodgkins lymphoma comprising administering to a subject in need thereof a therapeutically effective dose of an anti-CD80 antibody or a CD80-binding fragment thereof in an amount sufficient to induce apoptosis, effect lysis, or reduce cell growth of Hodgkin's Reed Sternberg cells within the lymphoma.

2. (canceled)

3. The method according to claim 1, wherein the anti-CD80 antibody or CD80-binding fragment thereof is a chimeric, humanized, or human antibody.

4. The method according to claim 1, wherein the anti-CD80 antibody binds a CD80 epitope bound by the antibody produced by ATCC Deposit No. HB-12119.

5. The method according to claim 1, wherein the anti-CD80 antibody or the fragment thereof competes for binding to CD80 with the antibody produced by ATCC Deposit No. HB-12119.

6. The method according to claim 1, wherein the anti-CD80 antibody comprises variable regions derived from variable regions of the antibody produced by ATCC Deposit No. HB-12119.

7. The method according to claim 6, wherein the anti-CD80 antibody comprises variable regions of the antibody produced by ATCC Deposit No. HB-12119.

8. The method according to claim 1, wherein the anti-CD80 antibody comprises complementarity determining regions (CDRs) of the antibody produced by ATCC Deposit No. HB-12119.

9. The method according to claim 1, wherein the anti-CD80 antibody is galiximab.

10-11. (canceled)

12. The method according to claim 1, wherein the subject is a human.

13. (canceled)

14. The method according to claim 1, wherein the Hodgkins lymphoma is of a type selected from the group consisting of Nodular Sclerosis Hodgkins lymphoma, Lymphocyte Predominant Hodgkins lymphoma, Mixed Cellularity Hodgkins lymphoma and Lymphocyte Depleted Hodgkins lymphoma.

15. The method according to claim 1, wherein the Hodgkins lymphoma is relapsed Hodgkins lymphoma or refractory Hodgkins lymphoma.

16. A method of inducing apoptosis or reducing cell growth of a Hodgkins Reed Sternberg cell comprising
contacting the Hodgkins Reed-Sternberg cell or a cell of a surrounding cellular infiltrate with an effective dose of an anti-CD80 antibody or a CD80-binding fragment thereof according to claim 1, to thereby induce apoptosis or inhibit cell growth of the Hodgkins Reed Sternberg cell.

17-24. (canceled)

25. The method according to claim 16, further wherein NFκB activation is downregulated in comparison to a control level of NFκB activation, or an activity of a pro-apoptotic molecule is upregulated in comparison to a control level of activity, or an activity of an anti-apoptotic molecule is downregulated in comparison to a control level of activity, or an amount of a cytokine secreted by the Hodgkins Reed Sternberg cell or by a cell in the surrounding infiltrate is reduced in comparison a control level of secreted cytokine.

26-33. (canceled)

34. A method of inducing lysis of a Hodgkins Reed Sternberg cell by antibody dependent cellular cytotoxicity comprising
contacting the Hodgkins Reed Sternberg cell with an effective dose of an anti-CD80 antibody or a CD80-binding fragment thereof according to claim 1, to thereby induce lysis of a Hodgkins Reed Sternberg cell by antibody dependent cellular cytotoxicity.

35-48. (canceled)

49. A method of treating Hodgkins lymphoma, comprising administering to a subject in need thereof a combination therapy comprising
(a) a therapeutically effective dose of an anti-CD80 antibody or a CD80-binding fragment thereof according to the method of claim 1, and
(b) an antibody selected from the group consisting of an anti-CD30 antibody, a CD30 binding fragment of the anti-CD30 antibody, an anti-CD40 antibody, a CD40-binding fragment of the anti-CD40 antibody, an anti-RANK antibody, a RANK-binding fragment of the anti-RANK antibody, an anti-RANKL antibody, a RANKL-binding fragment of the anti-RANKL antibody, an anti-TRAIL antibody, a TRAIL-binding fragment of the anti-TRAIL antibody, an anti-Notch antibody, a Notch-binding fragment of the anti-Notch antibody, an anti-LMP antibody, a LMP-binding fragment of the anti-LMP antibody, an anti-IL-13 antibody, a IL-13-binding fragment of the anti-IL-13 antibody, an anti-CD20 antibody, a CD20-binding fragment of the anti-CD20 antibody, an anti-CD52 antibody, a CD52-binding fragment of the anti-CD52 antibody, a CCR4 antibody, and a CCR4-binding fragment of the CCR4 antibody;
wherein (a) and (b) are administered concurrently or sequentially in either order.

50-57. (canceled)

58. The method according to claim 49, further comprising administering
(c) a chemotherapeutic agent,
wherein (a), (b), and (c) are administered concurrently or sequentially in any order.

59. A method of treating Hodgkins lymphoma comprising administering to a subject in need thereof a combination therapy comprising
(a) a therapeutically effective dose of an anti-CD80 antibody or a CD80-binding fragment thereof according to the method of claim 1; and
(b) a proteosome inhibitor or a histone deacytylase inhibitor;
wherein (a) and (b) are administered concurrently or sequentially in either order to thereby induce apoptosis or reduce cell growth of a Hodgkins Reed Sternberg cell.

60-67. (canceled)

68. The method according to claim 59, wherein the proteosome inhibitor is bortezomib.

69. The method according to claim 59, wherein the histone deacytylase inhibitor is suberoylanilide hydroximac acid.

70. The method according to claim 59, further comprising administering
(c) a chemotherapeutic agent,
wherein (a), (b), and (c) are administered concurrently or sequentially in any order.
